(19) United States
(12) Patent Application Publication
(10) Pub. No.: US 2018/0235882 A1
(43) Pub. Date: Aug. 23, 2018

(54) TOPICAL COMPOSITIONS COMPRISING CORTICOSTEROIDS

(71) Applicant: CADILA HEALTHCARE LIMITED, Ahmedabad, Gujarat (IN)

(72) Inventors: Shafiq Un Nabi SHEIKH, Ahmedabad (IN); Nilendu SEN, Thane (IN); Jitendrakumar Dasharathlal PATEL, Ahmedabad (IN); Abhijit CHATTERJEE, Ahmedabad (IN); Mukul JAIN, Ahmedabad (IN); Hardik Satish BODIWALA, Ahmedabad (IN); Biswajit SAHA, Thane (IN); Jay Shantilal KOTHARI, Ahmedabad (IN)

(21) Appl. No.: 15/756,196

(22) PCT Filed: Sep. 2, 2016
(86) PCT No.: PCT/IB2016/055262

§ 371 Date: Feb. 28, 2018

(30) Foreign Application Priority Data
Sep. 2, 2015 (IN) 3373/MUM/2015

(51) Int. Cl. A61K 9/107; A61K 31/573; A61K 45/06; A61K 9/06; A61K 9/00; A61K 47/26; A61K 47/10; A61K 47/32; A61K 47/02; A61K 47/44; A61K 47/38

(52) U.S. Cl. CPC A61K 9/1075; A61K 31/573; A61K 45/06; A61K 9/06; A61K 9/0014; A61K 47/38; A61K 47/10; A61K 47/32; A61K 47/02; A61K 47/44; A61K 47/26

(57) ABSTRACT

The present invention relates to the treatment of psoriasis and other skin disorders. More particularly, the present invention relates to compositions for treating such disorders, the compositions comprising submicron size droplets of corticosteroids and salts or esters thereof with oily excipients. The present invention also relates to process for the preparation of such compositions.

### FIELD OF THE INVENTION

The present invention relates to the treatment of psoriasis and other skin disorders characterized by redness, itching, flaking, scaling, and plaque-type growth. More particularly, the present invention relates to compositions for treating such disorders, the compositions comprising submicron size droplets of corticosteroids and salts or esters thereof with oily excipients either alone or dispersed in an aqueous medium. The present invention also relates to process for the preparation of such compositions.

### BACKGROUND OF THE INVENTION

Psoriasis is a chronic, immunologically based, inflammatory disease of the skin and joints, which has been estimated to affect 1% to 3% of the population. The estimated prevalence rates are mostly derived from general population health surveys (rather than from disease-specific studies) and from specific registries. Psoriasis is subject to flares and remissions and comes in many different variations and degrees of severity. It may be symptomatic throughout life and may be progressive with age or wax and wane in its severity. The published studies on the natural history of psoriasis are scarce, have inherent design limitations, and address few aspects important to psoriasis. Frequently, they are cross-sectional surveys usually involving a large series of patients with psoriasis gathered from hospitals and from dermatology clinics or patients identified from large-population, non-disease-specific surveys. Nevertheless, some studies provide data on specific aspects of the course of the disease, such as precipitating factors, comorbidity, incidence of cancer, and mortality.

The severity and course of psoriasis can vary greatly depending on the individual, but in general this condition recurs throughout the life of the individual causing significant psychological and social distress, and significantly impacting on quality of life. About 15% of people with psoriasis have joint inflammation that produces arthritis symptoms. This condition is called psoriatic arthritis. The exact mechanism which triggers the abnormal cell proliferation is not known, though researchers believe psoriasis occurs when faulty signals in the immune system cause skin cells to grow too rapidly. There are many treatments available but because of its chronic recurrent nature psoriasis is a challenge to treat.

Topical treatments performed by applying agents to the skin that slow down or normalize the excessive cell reproduction and reduce inflammation are usually the first line of defense in treating psoriasis and other skin disorders.

Topical corticosteroids are the most prescribed treatment for mild to moderate psoriasis and may also complement other psoriasis treatments for moderate to severe psoriasis. This is true for many of the other skin and mucus associated conditions above. Their efficacy is dependent on the ability of the corticosteroid molecule to activate corticosteroid receptors and the ability of the vehicle to deliver the active drug through the skin. The active ingredient is commonly formulated in a moisturizing base in several forms, including lotions and ointments, which provide a layer of oil on the surface of the skin, helping to prevent water from evaporating from the skin surface. This helps reduce the dryness, scaling and itching of skin conditions such as eczema.

If corticosteroids are used on large areas of skin or mucous membranes for long-term, there are chances that there will be very high level of corticosteroids in blood. This increases the risk of local side effects such as skin thinning, and of systemic side effects, such as adrenal suppression, where the adrenal glands become unable to regulate hormones being released in the body; and tachyphylaxis, where the body develops immunity to a certain treatment regimen. Moreover, the need for long-term application may be a daunting task for many patients. Poor adherence to treatment over time, due to an undesirable vehicle, may result in poor treatment outcomes.

Unsatisfactory treatment of the psoriasis and related disorders has a considerable adverse impact on the patient's quality of life, very often the patients complaining about the messiness of the topical agents used. Therefore, it is essential to have a proper formulation delivering system for topical steroids to avoid unnecessary side effects and ensure a positive treatment outcome. 21-chloro-9-fluoro-11β, 17-dihydroxy-16β-methylpregna-1,4-diene-3,20-dione propionate (clobetasol propionate) is a very potent corticosteroid and is one of the most common topical therapies used for psoriasis. The efficiency, tolerability, and applicability of topical agents are directly related to employed vehicles. Thus, to achieve optimum topical therapy, vehicle composition, and its physical action on the skin is important. Common vehicles are complex mixtures consisting of diverse ingredients belonging to various groups, i.e. hydrophilic and lipophilic bases, emulsifiers, gel-forming agents, preservatives, and antioxidants. The proper choice of the vehicle, with respect to the properties of the incorporated active ingredient, is of paramount importance to maximize the therapeutic effect at the site of application without any adverse effects.

Clobetasol propionate is commercially available in compositions for topical application in the form of lotion, spray, cream or shampoo, in a weight concentration of 0.05%. Moreover, various topical pharmaceutical compositions comprising clobetasol propionate have been proposed in the prior art, claiming the use of particular carriers or excipients.

U.S. Pat. No. 5,972,920 claims a formulation characterized by a carrier compound formed of a combination of two components in a volume ratio of about 50/50, wherein the first carrier component is selected from the group consisting essentially of ethyl alcohol and isopropyl alcohol and the second carrier component is selected from the group consisting essentially of isopropyl myristate, isopropyl palmitate, octyl palmitate, octyl isononanoate, and isocetyl stearate. The patent covers the marketed formulation “CLOBEX” spray. The formulation also comprises an anionic surfactant.

The patent discloses the use of alcohol in an amount of about 50% w/w of the composition. This high concentration of alcohol causes dryness and itching at the site of application. However, in contrast, the present invention comprises alcohol in an amount of not more than 10% w/w of the composition and hence provides benefit over marketed composition in terms of dryness and itching.

U.S. Pat. No. 8,992,994 discloses stable topical pharmaceutical composition comprising: (a) 0.01-0.5% clobetasol or a salt or ester thereof; (b) 4-8% salicylic acid; (c) 1-5% lactic acid; (d) 1-5% polyoxyethylene (20) sorbitan monooleate; (e) 3-7% of alcohol; (f) 3-7% of lipids; (g) 7-12% of oils; (h) 0.05-0.5% of butylated hydroxytoluene; (i) 0.5-3% of a carbomer; (j) one or more pH adjusting agents; and (k) water; wherein the composition comprises a particle size of about 250 nm and a pH of about 5.87. The patent specifically discusses about the combination of clobetasol and salicylic acid along with lactic acid. Further, all the disclosures of the patent pertains to gel formulation, specifically.

International PCT Publication No. WO 2006115987 provides a method for treating psoriasis by spraying a pharmaceutical composition containing an effective amount of clobetasol propionate onto the skin with psoriasis, using a daily treatment for at least 4 weeks. The preferred composition is a spray formulation of clobetasol propionate 0.05%, containing alcohol, isopropyl myristate, an anionic surfactant such as sodium lauryl sulfate, and optionally an antimicrobial compound such as an antifungal compound like undecylenic acid.

U.S. Pat. No. 6,579,512 refers to a propellant containing pharmaceutical topical spray composition of a corticosteroid, consisting essentially of clobetasol propionate and isopropyl myristate. The composition also comprises an alcohol.

According to U.S. Pat. No. 5,990,100, isopropyl myristate is an active agent for treating psoriasis and can be combined as the first active agent with a known anti-psoriatic agent (second active agent) giving a more effective multi-active-agent composition. The known anti-psoriatic agent can be a corticosteroid. The composition also comprises sodium lauryl sulphate and Polysorbate 80 (polyoxyethylene (20) sorbitan monooleate) as emulsifiers (or dispersants or surfactants) in a water/ethanol vehicle.

International PCT Publication No. WO 2004093722 reports a composition comprising at least one polyhydroxy lactone or polyhydroxy acid compound selected from a polyhydroxy-aldonic acid, a polyhydroxy-aldonic lactone, a polyhydroxy-alduronic acid, a polyhydroxy-alduronic lactone, a polyhydroxy-aldaric acid, polyhydroxy-aldaric lactone, and an organic acid lactone having two or more hydroxyl or ketohydroxyl groups. Active agents, including clobetasol propionate, may be incorporated into the composition.

International PCT Publication No. WO 2007104895 and WO 2007104897 disclosed oil-in-water (O/W) anti-inflammatory emulsions, containing: a) a therapeutically effective amount of at least one steroidal anti-inflammatory agent, notably clobetasol propionate; b) a pro-penetrating system which includes at least one pro-penetrating glycol and at least one other pro-penetrating agent; a polymeric or non-polymeric emulsifier or one gelling agent In both the cases preferred pro-penetration system are: propylene glycol and isosorbide dimethyl; propylene glycol and ethanol; propylene glycol, monoethylether of diethylene glycol and propylene glycol laurate; propylene glycol and methylpyrrolidone; propylene glycol, isosorbide dimethyl and ethanol; propylene glycol, methylpyrrolidone and oleic alcohol.

International PCT Publication No. WO 2007020349 discloses a topically applicable oil-in-water emulsion containing at least one biologically active agent and also comprising: a) a fatty phase, the amount thereof ranging from about 35% to 50% by weight; b) from about 1% to 15% by weight of a nonionic emulsifying system; c) from about 1% to 30% by weight of at least one pro-penetrating agent; and d) from about 5% to 50% by weight of water. The fatty phase is emulsified by means of a nonionic emulsifying system (nonionic surfactant) with a predominant hydrophilic fraction.

EP 1360958 relates to a liposomic formulation of clobetasol propionate, wherein the clobetasol propionate is encapsulated in liposomes and also contains phospholipid; a lipophilic additive; anti-oxidant agents; an organic solvent; a buffer agent to maintain the pH level of the water component at 5 to 7.5; preservative substances; gelling agents; water.

U.S. Publication No. 20100249060 discloses a pharmaceutical composition for topical use, comprising 0.005% to 0.05% by weight of 17-clobetasol propionate in an aqueous vehicle comprising propylene glycol as a solvent and moisture-retaining agent, and macrogol-glycerol hydroxystearate as a non-ionic emulsifier.

Some other documents disclose the use of clobetasol in combination with at least one other active ingredient, such as calcitriol (Vitamin D), for example, International PCT Publication No. WO 2008110815, EP 1854466, EP 1875916, U.S. Publication No. 2005281850, U.S. Publication No. 2006009426 and FR 2848454; a progesterone derivative (EP 1473300); a prostaglandin (International PCT Publication No. WO 03092617); or tazarotene (CA 2282682).

As shown above, dermatological corticosteroids and in particular, clobetasol propionate have been provided in a variety of topical formulations, such as creams, lotions, gels, and the like in attempts to increase the delivery efficiency. However, while preparing topical formulations of corticosteroids, formulators usually encountered various problems. A first problem in the preparation of dermatological compositions is delivery efficiency of the composition. A second problem is related to stability, i.e. both of the active ingredient and of the auxiliary components in the formulation and in turn formulation stability. A further problem is tolerability, in particular with respect to excipients that would cause irritation.

Further, topical corticosteroids should be absorbed through the skin and travel into the bloodstream. This may constitute another problem, in particular when widespread areas of body is treated, and can provoke unwanted side effects, like hypothalamic pituitary adrenal (HPA) axis suppression and related side effects. Numerous studies have demonstrated the potential for HPA axis suppression from most high-potency topical agents. As little as 2 gm per day of clobetasol propionate, 0.05%, can cause a decrease in morning cortisol after only a few days. In fact, deaths have been reported from the misuse of high-potency topical corticosteroids and resultant Addisonian crisis. Because of the potential for serious local and systemic side effects with prolonged use, most super potent topical corticosteroids carry some type of prescription limit in their package inserts.

Therefore, the factors that contribute to the potential for increased suppression of HPA axis includes amount applied, percentage of body surface covered, frequency of application, application to thin skin or naturally occluded areas (i.e., axillae), age of patient, skin quality (i.e., diseased), and potency of steroid.

Usually ointments are commonly prescribed for psoriasis, in part because of the perception that they are more potent and in part because of the perception that moisturizing psoriasis plaques is inherently beneficial. Nevertheless, ointment-based vehicles are among the least appealing to patients due to their messiness and greasy feel. Bothersome aspects of the vehicle likely reduce patients' adherence to treatment. Discontinuation of topical steroids should be obviated, to avoid a psoriasis “rebound”.

Topical clobetasol propionate is currently one of the most used treatments for psoriasis and its safety and efficacy is well defined in the medical literature. However, current formulations of clobetasol propionate present several disadvantages. Cream and ointment are greasy and difficult to apply on large areas, and thus negatively impact the treatment compliance and quality of life. The use of other pharmaceutical forms is restricted to short periods of time due to the risk of side effects and due to high concentration of alcohol and penetration enhancers.

The inventors of the present invention surprisingly found that topical pharmaceutical compositions comprising low dose of clobetasol propionate in submicron size droplets with oily excipients along with viscosity modifying agent, wherein the viscosity modifying agent provides increase in viscosity of the emulsion such that the composition is retained in the vicinity of the area of application for a sufficient period of time to permit an increased uptake of the physiologically active agent at that site and in order help in decreasing the dose, which in turn help in decreasing the above mentioned side effects.

Further, the use of viscosity modifying agent also decreases the solvent evaporation rate and hence helps retain moisture for longer duration. At the same time, the composition showed to have a very good chemical stability, consequently providing the product a surprisingly long durability. Further, the inventors of present invention surprisingly found that a topical pharmaceutical composition of the present invention is capable of holding surprisingly low concentrations of clobetasol propionate and provide therapeutic efficacy in comparison to marketed formulations of high concentration.

### SUMMARY OF THE INVENTION

In one general aspect, the present invention provides pharmaceutical compositions for topical application comprising low dose of corticosteroids and salts or esters thereof in submicron size droplets with oily excipients either alone or dispersed in an aqueous medium, wherein the corticosteroids and salts or esters thereof is present in concentrations of about 0.001% to about 0.05% w/w.

Embodiments of the pharmaceutical composition may include one or more of the following features. The corticosteroids and salts or esters thereof may be present in concentrations of about 0.005 to 0.025% w/w, preferably, about 0.01% w/w to about 0.02% w/w. The droplet size may be below one micron, for example, in the range of about 0.05 to 0.25 microns. The composition may include one or more viscosity modifying agents. The aqueous medium may include one or more emulsifying agents.

In another general aspect, the present invention provides a process for the preparation of pharmaceutical compositions comprising low dose of corticosteroids and salts or esters in concentrations of about 0.005 to 0.025% w/w, wherein the process of preparation comprising following steps:

a) combining an oily phase comprising corticosteroids and salts or esters thereof in concentrations of about 0.005 to 0.025% w/w with other pharmaceutically acceptable excipients with an aqueous phase to form an emulsion;

b) reducing the droplet size of emulsion of step a) to a droplet size having D₉₀ particle size of less than 250 nm; and

c) mixing other pharmaceutically acceptable excipients to the emulsion obtained in step b) and converting it into a suitable finished dosage form.

In a further more detailed aspect, the present invention relates to a pharmaceutical composition comprising low dose of clobetasol and salts or esters thereof in submicron size droplets with oily excipients dispersed in an aqueous medium, said aqueous medium comprising of an emulsifying agent and at least one viscosity modifying agent, wherein the clobetasol and salts or esters thereof is present in concentrations of about 0.005 to 0.025% w/w.

In a further more detailed aspect, the present invention relates to a pharmaceutical composition comprising low dose of clobetasol and salts or esters thereof in submicron size droplets with oily excipients dispersed in an aqueous medium, said aqueous medium comprising of an emulsifying agent and at least one viscosity modifying agent, wherein the clobetasol and salts or esters thereof is present in concentrations of about 0.01% w/w to about 0.02% w/w.

In a further more detailed aspect, the present invention relates to a pharmaceutical spray composition comprising low dose of clobetasol and salts or esters thereof in submicron size droplets with oily excipients dispersed in an aqueous medium, said aqueous medium comprising of an emulsifying agent and at least one viscosity modifying agent, wherein the clobetasol and salts or esters thereof is present in concentrations of about 0.005 to 0.025% w/w.

Another aspect of the present invention provides pharmaceutical composition of corticosteroids and salts or esters thereof for topical administration for use for the prevention and/or treatment of a skin disease, which are characterized by comparative efficacy of therapeutic agent into the derma with respect to marketed formulations of high concentration, wherein the corticosteroids and salts or esters thereof is present in concentrations of about 0.005 to 0.025% w/w.

In a further more detailed aspect, the present invention relates to a pharmaceutical composition comprising low dose of corticosteroids and salts or esters thereof in submicron size droplets, for use in the treatment of psoriasis and other inflammatory skin and mucous membrane disorders including psoriasis, eczema, atopic dermatitis, contact dermatitis and seborrhoeic dermatitis and other forms of dermatitis, wherein the corticosteroids and salts or esters thereof is present in concentrations of about 0.005 to 0.025% w/w.

The details of one or more embodiments of the present invention are set forth in the description below. Other features, objects and advantages of the invention will be apparent from the description.

### BRIEF DESCRIPTION OF THE DRAWINGS

FIG. 1 shows the effects of clobetasol formulations on ear thickness on day 6 (Composition of Example-2 & 3)

FIG. 2 shows the effects of clobetasol formulations on ear weight (Composition of Example-2 & 3)

FIG. 3 shows the effects of clobetasol formulations on erythema score on day 6 (Composition of Example-2 & 3)

FIG. 4 shows the effects of clobetasol formulations on scaling score on day 6 (Composition of Example-2 & 3)

FIG. 5 shows the effects of clobetasol formulations on spleen weight per gram of body weight (Composition of Example-2 & 3)

FIG. 6 shows the effects of clobetasol formulations on serum corticosterone level (Composition of Example-2 & 3)

FIG. 7 shows the effects of clobetasol formulations on ear thickness on day 6 (Composition of Example-1)

FIG. 8 shows the effects of clobetasol formulation on ear weight (Composition of Example-1)

FIG. 9 shows the effects of clobetasol formulation on erythema score on day 6 (Composition of Example-1)

FIG. 10 shows the effects of clobetasol formulation on scaling score on day 6 (Composition of Example-1)

FIG. 11 shows the effects of clobetasol formulation on spleen weight per gram of body weight (Composition of Example-1)

FIG. 12 shows the effects of clobetasol formulation on serum corticosterone level (Composition of Example-1)

FIG. 13 shows the effects of clobetasol formulation on ear thickness on day 7 (Composition of Example-1)

FIG. 14 shows the effects of clobetasol formulation on ear weight (Composition of Example-1)

FIG. 15 shows the effect of various clobetasol formulations in IMQ induced psoriasis model in C57BL/6 mice (Example-5).

FIG. 16: Characterization of clobetasol propionate spray 0.01% submicron formulations (Example-8) using transmission electron cryo-microscopy (cryoTEM) and sizing analysis.

FIG. 17: Characterization of clobetasol propionate nanoemulsion 0.01% submicron formulations (Example-12) using transmission electron cryo-microscopy (cryoTEM) and sizing analysis.

FIG. 18: Area equivalent diameter (AED) metric distributions for clobetasol spray (Example-8) and nanoemulsion submicron formulations of 0.01% (Example-12). The top are the probability distribution function (PDF) and the bottom is the cumulative probability distribution (CDF). Plot axes ranges are chosen to include all data.

FIG. 19. Max feret diameter metric distributions of clobetasol spray (Example-8) and nanoemulsion submicron formulations of 0.01% (Example-12). The top is the probability distribution function (PDF) and the bottom is the cumulative probability distribution (CDF). Plot axes ranges are chosen to include all data.

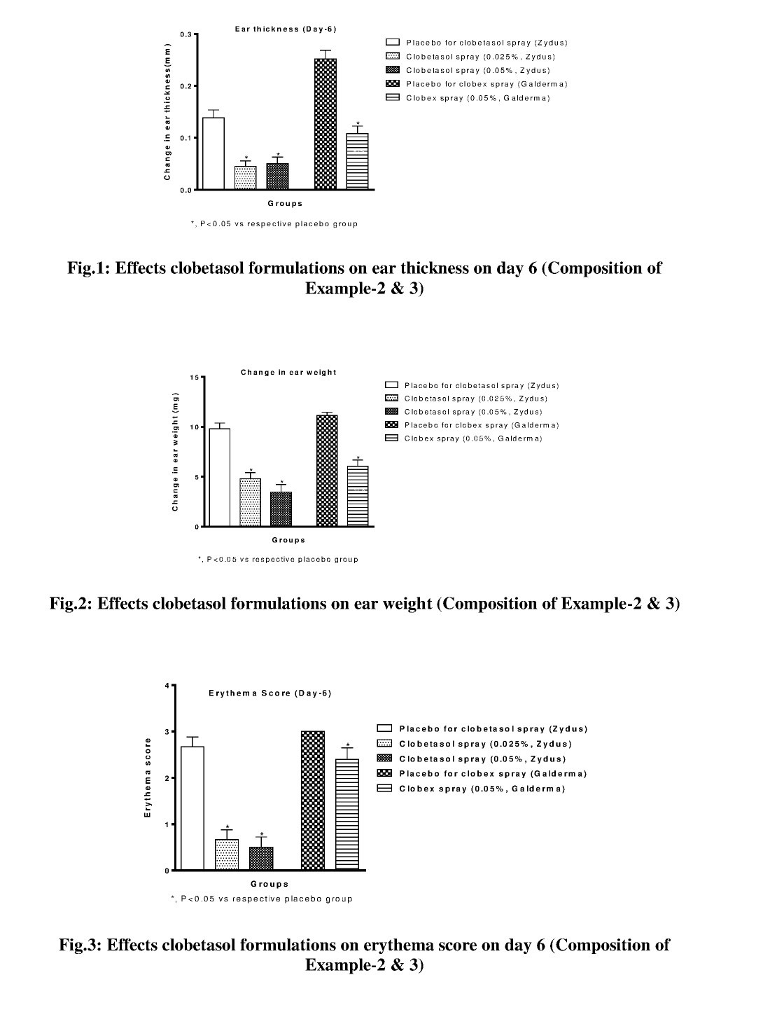

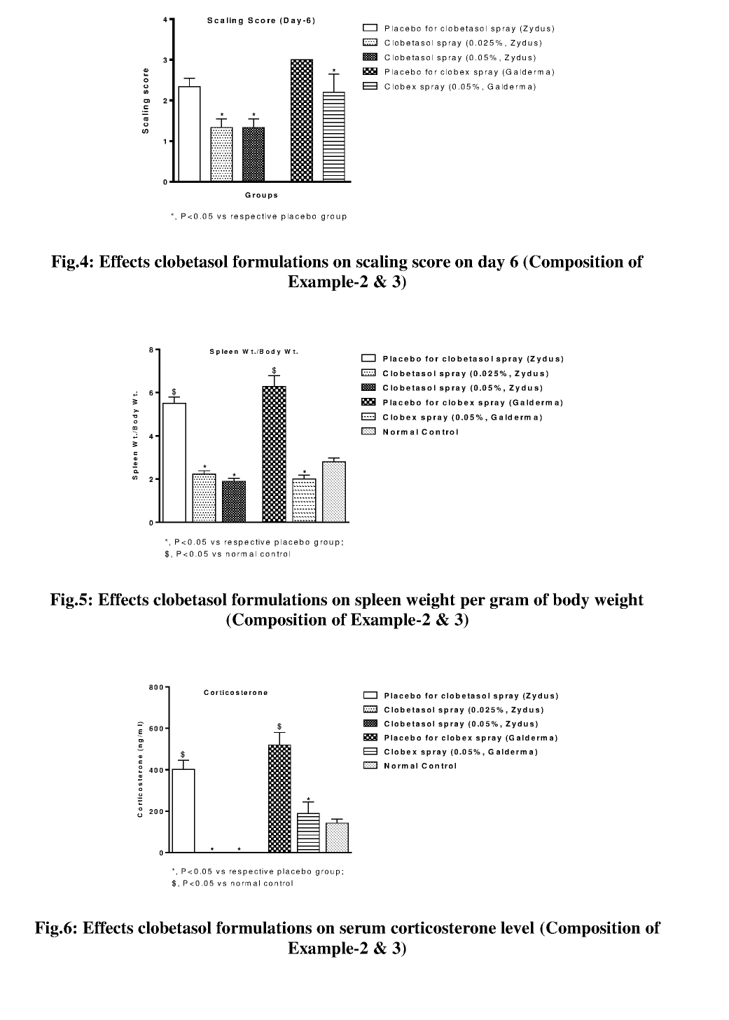

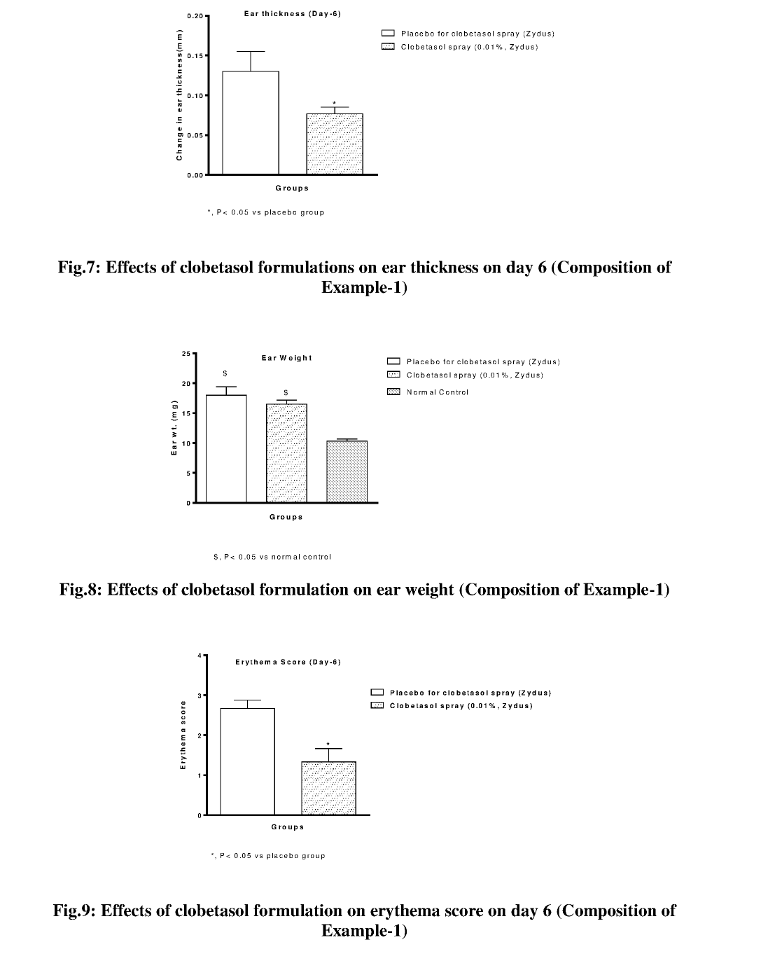

### DETAILED DESCRIPTION OF THE INVENTION

Chronic skin conditions, such as psoriasis, however often require long periods of treatment, to manage such conditions. Therefore, it would be desirable to have a superpotent corticosteroid formulation with a reduced incidence and/or severity of systemic side effects so that therapy can be continued for longer durations.

The applicant has developed a method and a composition for topical administration which allows appropriate delivery of active agents and reduces the risk of undesirable side effects in a patient. Further, applicants of present invention have surprisingly found that the present pharmaceutical composition comprising low dose of corticosteroids and salts or esters thereof in submicron size droplets, provides the better efficacy of the drug. In the present invention, the efficacy of the corticosteroid is maintained, even when the concentration of the corticosteroid is markedly reduced.

Due to the much reduced concentration of the corticosteroid in the formulation, reduced amounts of corticosteroid are available to enter the systemic circulation and, therefore, the tendency of such formulations to cause undesirable systemic side effects should likewise be reduced. Thus, the formulations and methods of this application are especially useful for the treatment of chronic or recalcitrant skin diseases, such as psoriasis, due to the concerns regarding safety when topical corticosteroids are used in multiple course treatments over time.

In one embodiment, there is provided a pharmaceutical compositions for topical application comprising low dose of corticosteroids and salts or esters thereof in submicron size droplets with oily excipients either alone or dispersed in an aqueous medium, wherein the corticosteroids and salts or esters thereof is present in concentrations of about 0.001 to 0.05% w/w.

In one embodiment, there is provided a pharmaceutical compositions for topical application comprising low dose of corticosteroids and salts or esters thereof in submicron size droplets with oily excipients either alone or dispersed in an aqueous medium, wherein the corticosteroids and salts or esters thereof is present in concentrations of about 0.005 to 0.025% w/w.

The term “submicron” is used herein to mean a size of about 0.05 to 0.30 μm. Thus, submicron droplets of these sizes would be smaller than those of a classical macroemulsion, which has droplet sizes of above about 0.5 μm.

In another embodiment, there is provided a pharmaceutical compositions for topical application comprising low dose of corticosteroids and salts or esters thereof in submicron size droplets with oily excipients either alone or dispersed in an aqueous medium, wherein composition further comprises at least one viscosity modifying agent, wherein the corticosteroids and salts or esters thereof is present in concentrations of about 0.005 to 0.025% w/w.

The term “viscosity modulating agent” is used herein to refer to a component of the composition which alters the viscosity of the overall resulting composition. The nature of the viscosity modulating agent depends not only on the agent itself, but also the proportion in which it is present and the presence or absence of other components. For example, a gelling agent may act as a viscosity modulating agent providing that an activator for that gelling agent is present. For example, hydroxypropylmethylcellulose (HPMC) may be used in a composition with an activator. A suitable activator would be alkali.

Typically, the composition appears like a lotion. In this context, “lotion” is used in its broad descriptive sense, rather than the more specific formulatory sense which refers to a mixed phase or suspension of active. The composition of the invention is often a solution, but with increased viscosity so that its viscosity is more similar to that usually associated with a lotion. The viscosity of the composition is preferably greater than that of water and less than 500 centipoise.

Further, the viscosity modulating agent will commonly be a thickening agent or a gelling agent. It will often be used to increase the viscosity of the composition containing a solution of the physiologically active agent in the volatile solvent. The purpose of the viscosity modulating agent is to increase the viscosity of the solution such that the composition is retained in the vicinity of the area of application for a longer period of time so as to permit increased uptake of the physiologically active agent at that site. The viscosity modulating agent will typically increase the viscosity to about that of a typical lotion, but not to the point where the composition becomes a gel. Typically, the viscosity in different embodiments is in the range of 50 to 300 centipoise. More preferably, the viscosity is in the range of 10 to 200 centipoise.

Suitable viscosity modifying agent may include one or more of cellulose polymer, a cellulose derivative, crosslinked acrylic acid polymers such as the acrylamide/sodium acryloyldimethyltaurate copolymer-isohexadecane-polysorbate 80 or “carbomer” family of polymers, e.g., carboxypolyalkylenes that may be obtained commercially under the CARBOPOL® such as CARBOPOL 940 or CARBOPOL 974P, polyvinyl alcohol, poloxamers, polysaccharides, polyvinyl pyrrolidone, xanthan gum, and the like.

Preferably viscosity modifying agent may be present in an amount of from about 0.01 to 5%, and is preferably from about 0.1 to 4%, more preferably from about 0.1 to 3% w/w of the composition.

In another embodiment, there is provided a pharmaceutical composition of low dose corticosteroids and salts or esters thereof for topical administration for use for the prevention and/or treatment of a skin disease, which have a high level moisturizing activity. To provide high level moisturizing activity, viscosity modulating agents plays an important role. Desirably, a viscosity modulating agents inhibits the solvent evaporation rate so as to retain moisture for longer duration at the site of application.

In another embodiment, there is provided a pharmaceutical composition comprising low dose of corticosteroids and salts or esters thereof in submicron size droplets with oily excipients dispersed in an aqueous medium, said aqueous medium comprising of an emulsifying agent and at least one viscosity modifying agent, wherein the corticosteroids and salts or esters thereof is present in concentrations of about 0.005 to 0.025% w/w.

In another embodiment, there is provided a pharmaceutical composition comprising low dose of corticosteroids and salts or esters thereof in submicron size droplets with oily excipients dispersed in an aqueous medium, said aqueous medium comprising of an emulsifying agent and at least one viscosity modifying agent, wherein the corticosteroids and salts or esters thereof is present in concentrations of about 0.01% w/w to about 0.02% w/w.

The term “emulsifying agent” is used herein to refer to a component of the composition which helps in dispersion of oily excipients in an aqueous medium. Emulsifying agent constitute an important component of the formulations, as they lend to the stability of the emulsion as a whole by coating the oil droplets. The “emulsifying agent” may be an emulsifiers or surfactants.

Preferred emulsifiers include a phospholipid compound or a mixture of phospholipids. Suitable components include lecithin; MONTANOL-68, EPICURON 120 (Lucas Meyer, Germany) which is a mixture of about 70% of phosphatidylcholine, 12% phosphatidylethanolamine and about 15% other phospholipids; OVOTHIN 160 (Lucas Meyer, Germany) which is a mixture comprising about 60% phosphatidylcholine, 18% phosphatidylethanolamine and 12% other phospholipids; a purified phospholipid mixture; LIPOID E-75 or LIPOID E-80 (Lipoid, Germany) which is a phospholipid mixture comprising about 80% phosphatidylcholine, 8% phosphatidylethanolamine, 3.6% non-polar lipids and about 2% sphingomyelin. Purified egg yolk phospholipids, soybean oil phospholipids or other purified phospholipid mixtures are useful as this component.

The surfactant chosen should preferably be non-ionic to minimize irritation, and one skilled in the art can conduct tests to routinely select specific surfactants for this purpose. Generally, the surfactant is a non-ionic alkylene oxide condensate of an organic compound which contains one or more hydroxyl groups. For example, ethoxylated and/or propoxylated alcohol or ester compounds or mixtures thereof are commonly available and are well known to those skilled in the art. Suitable surfactants include, but are not limited to, polyoxyethylene sorbitan ester e.g. polyoxyethylenesorbitan monooleate such as TYLOXAPOL; POLOXAMER 4070; POLOXAMER 188; POLYOXYL 40 Stearate; EMULFOR EL-620, POLYSORBATE 80, and POLYSORBATE 20, as well as various compounds sold under the trade name TWEEN (ICI American Inc., Wilmington, Del., U.S.A.), PLURONIC F-68 (trade name of BASF, Ludwigshafen, Germany for a copolymer of polyoxyethylene and polyoxypropylene). Emulsifying agents are, essentially, surfactants.

Preferably Emulsifying agents may be present in an amount of from about 0.1% to 10% w/w of the composition.

In another embodiment, there is provided a process to prepare pharmaceutical composition comprising low dose of corticosteroids and salts or esters thereof in submicron size droplets with oily excipients dispersed in an aqueous medium, said aqueous medium comprising of an emulsifying agent and at least one viscosity modifying agent, wherein the corticosteroids and salts or esters thereof is present in concentrations of about 0.005 to 0.025% w/w.

In another embodiment, there is provided a process to prepare pharmaceutical composition comprising low dose of corticosteroids and salts or esters thereof in submicron size droplets with oily excipients dispersed in an aqueous medium, said aqueous medium comprising of an emulsifying agent and at least one viscosity modifying agent, wherein the corticosteroids and salts or esters thereof is present in concentrations of about 0.01% w/w.

In further embodiment, process to prepare the pharmaceutical composition of the invention comprises:

a) combining an oily phase comprising corticosteroids and salts or esters thereof in concentrations of about 0.005 to 0.025% w/w with other pharmaceutically acceptable excipients with an aqueous phase to form an emulsion;

b) reducing the droplet size of emulsion of step a) to a droplet size having D₉₀ particle size of less than 250 nm; and

c) mixing other pharmaceutically acceptable excipients to the emulsion obtained in step b) and converting it into a suitable finished dosage form.

In further embodiment, process to prepare the pharmaceutical composition of the invention comprises:

a) combining an oily phase comprising corticosteroids and salts or esters thereof in concentrations of about 0.01% w/w with other pharmaceutically acceptable excipients with an aqueous phase to form an emulsion;

b) reducing the droplet size of emulsion of step a) to a droplet size having D₉₀ particle size of less than 250 nm; and

c) mixing other pharmaceutically acceptable excipients to the emulsion obtained in step b) and converting it into a suitable finished dosage form.

Among the suitable corticosteroids there may be included: hydrocortisone; hydrocortisone acetate; hydrocortisone butyrate; hydrocortisone valerate; triamcinolone acetonide; fluocinolone acetonide; 16-hydroxy prednisolone-16α, 17-acetonide; fluohydrocortisone; 1-dehydroxycortisone; β-methasone; 9α,11β-dicloro-6α-fluoro-21-hydroxy-16α,17α-isopropylidenedioxypregna-1, 4-diene-3,20-dione; 9α-fluoro-11β,17α-21-trihydroxy-16β-methylpregna-1, 4-diene-3,20-dione; 9α-fluoro-11β,21-dihydroxy-16β-methyl-17α-valeroxy-pregna-1,4-diene-3,20-dione; 17α,21-dihydroxypregn-4-ene-3,11,20-trione; 17α-hydroxy-21-acetoxypregn-4-ene-3,11, 20-trione; 21-hydroxypregn-4-ene-3, 20-dione; 21-acetoxypregn-4-ene-3, 20-dione; 21-divaloxypregn-4-ene-3, 2-dione; 9α-fluoro-11β, 17α,21-trihydroxy-16α-methylpregna-1,4-diene-3, 20-dione; 6α,90-difluoro-11β, 17α-dihydroxypregna-1,4-diene-3, 20-dione; 6α-fluoro-11β,21-dihydroxy-16α-17α-isopropylidenedioxypregn-4-ene-3, 20-dione; 11β,17α-dihydroxy-21-acetoxypregna-4-ene-3, 20-dione; 6α-methyl-11β,17α,21-trihydroxypregna-1,4-diene-3, 20-dione; 6α-methyl-11β,17α-dihydroxy-21-acetoxy-pregna-1,4-diene-3, 20-dione; 6α-fluoro-11β,17α,21-trihydroxy-16α-methylpregna-1, 4-diene-3, 20-dione; 6α-fluoro-11β,17α-dihydroxy-16α-methyl-21-acetoxy-pregna-1,4-diene-3, 20-dione; 6α-fluoro-11β,hydroxy-16α, 17α-isopropylidenedioxy-21-acetoxypregna-1,4-diene-3,20-dione; 11β,17α,21-trihydroxypregna-1,4-diene-3,20-dione; 11β,17α-dihydroxy-21-acetoxypregna-1,4-diene-3, 20-dione; 17α,21-dihydroxypregna-1,4-diene-3,11,20-trione; 17α,hydroxy-21-acetoxypregna-1,4-diene-3-11,20-trione; 9α-fluoro-11β,16β,17α,21-tetrahydroxypregna-1,4-diene-3, 20-dione; 9α-fluoro-11β,16α, 17α-trihydroxy-21-acetoxypregna-1,4-diene-3, 20-dione; 6α,9α-difluoro-11β,21-dihydroxy-16α-methyl-17α-valeroxypregna-1,4-diene-3, 20-dione; 6α, 9α-difluoro-11β,17α,21-trihydroxy-16α-methyl pregna-1,4-diene-3, 20-dione; 6α,7α-difluoromethylene-11β,17α,21-trihydroxypregna-4-ene-3, 20-dione; 6α, 9α-difluoro-11β-hydroxy-16α, 17α-isopropylidenedioxy-21-chloropregna-1,4-diene-3, 20-dione; 9α, 11-dichloro-6α,21-difluoro-16α, 17α-isopropylidenedioxypregna-1,4-diene-3,20-dione, and 9α, 11β,21-trichloro-6α-fluoro-16α, 17α-isopropylidenedioxypregna-1,4-diene-3, 20-dione; clobetasol and salts or esters thereof.

Preferably corticosteroid is clobetasol and salts or esters thereof. More preferably corticosteroid is clobetasol.

In another embodiment, there is provided a pharmaceutical composition comprising low dose of clobetasol and salts or esters thereof in submicron size droplets with oily excipients dispersed in an aqueous medium, said aqueous medium comprising of an emulsifying agent in concentrations about 0.1% to 10% w/w and at least one viscosity modifying agent in concentrations about 0.01 to 5% w/w, wherein the clobetasol and salts or esters thereof is present in concentrations of about 0.005 to 0.025% w/w.

In another embodiment, there is provided a pharmaceutical composition comprising low dose of clobetasol and salts or esters thereof in submicron size droplets with oily excipients dispersed in an aqueous medium, said aqueous medium comprising of an emulsifying agent in concentrations about 0.1% to 10% w/w and at least one viscosity modifying agent in concentrations about 0.01 to 5% w/w, wherein the clobetasol and salts or esters thereof is present in concentrations of about 0.01% w/w.

In another embodiment, the present invention may comprise another active agent in combination with clobetasol and salts or esters thereof; the active agent may be one or more of the following: a steroid, non-steroidal anti-inflammatory drug, antibiotic, tranquilizer, sedative, anti-histaminic, antifungal, antibacterial, antiviral, disinfectant, antipsoriatic agent or a local anesthetic. More preferably, the active ingredient may be antipsoriatic agent. The anti-psoriatic agents useful for the purpose of the present invention may be selected from, but not limited to topical steroids such as Mometasone, Triamcinolone, Flucinonide, Desoximetasone, Alclometasone, Hydrocortisone, Diflorasone, Flurandrenolide, Amcinonide, Prednicarbate, Desonide, Halcinonide, Clocortolone; Psoralens such as Methoxsalen; Antipsoriatics such as Acitretin; Immunosuppressive agents such as Alefacept, Cyclosporin; Glucocorticoid such as Triamcinolone, Prednisone; Topical antipsoriatics such as Calcipotriene, Anthralin, Resorcinol, Betamethasone, Calcipotriene, Tazarotene; Antirheumatics, TNF inhibitors such as Etanercept, Adalimumab, Efalizumab, Ustekinumab; Topical emollients such as Ammonium lactate, Urea; Corticotropin and coal tar; glycosaminoglycan such as hyaluronic acid and derivatives thereof; antioxidants such as resveratrol.

In another embodiment, there is provided a pharmaceutical composition of corticosteroids and salts or esters thereof in combination with hyaluronic acid and derivatives thereof. The inventor of the present invention explored the possibility of insuring a sufficient therapeutic effect with a topical preparation containing a reduced concentration of corticosteroids and found that when hyaluronic acid is incorporated in a topical therapeutic composition comprising corticosteroids and salts or esters thereof in such a reduced concentration, a therapeutic effect comparable to or even surpassing the effect obtainable with the conventional topical preparation containing the usual clinical dose of the corticosteroids and salts or esters thereof can be obtained. Since hyaluronic acid is not irritating or toxic, the topical dermatological composition of the invention can be used in the same manner as the conventional corticosteroids and salts or esters thereof for external use. Further, according to the invention hyaluronic acid helps in both inflammatory conditions and to degeneration caused by treatment with corticosteroid preparations. It is thus clear that a sufficiently high molecular weight of the hyaluronic acid is alleged to counteract side effects that might be caused by corticosteroids such as pain, swelling, and lameness. The amount of hyaluronic acid and derivatives thereof may range from about 0.005 to 0.1% w/w of the total weight of the composition.

In yet another embodiment there is provided a pharmaceutical composition of corticosteroids and salts or esters thereof in combination with resveratrol. The present combination provides a method of improving or preventing the condition of wrinkled, lined, dry, flaky, aged or photodamaged skin caused by application of topical corticosteroid and hence improving skin thickness, elasticity, flexibility, radiance, glow and plumpness. Compositions of the invention are intended for topical application to mammalian skin which is already dry, flaky, lined, wrinkled, aged, photodamaged, or the inventive compositions may be applied prophylactically to reduce the deteriorative changes. The amount of resveratrol may range from about 0.01 to 0.5% w/w of the total weight of the composition.

In another embodiment, there is provided a pharmaceutical composition of clobetasol and salts or esters thereof for topical administration for use for the prevention and/or treatment of a skin disease, which are characterized by an efficacious penetration of therapeutic agent into the derma without any significant systemic absorption.

The pharmaceutical formulation of the present invention may further contain one or more suitable solvents. Examples of such solvents include, but are not limited to, water, tetrahydrofuran, isopropyl alcohol, propylene glycol, liquid petrolatum, ether, petroleum ether, alcohols, e.g., methanol, ethanol and higher alcohols, aromatics, e.g., benzene and toluene, alkanes, e.g., pentane, hexane and heptane, ketones, e.g., acetone and methyl ethyl ketone, chlorinated hydrocarbons, e.g. chloroform, carbon tetrachloride, methylene chloride and ethylene dichloride, acetates, e.g., ethyl acetate, oils, e.g., isopropyl myristate, diisopropyl adipate and mineral oil and the like and mixtures thereof.

Preferably solvent used in present invention is alcohol e.g., methanol, ethanol and higher alcohols. Preferably alcohol may be present in an amount of from about 0.1% to 10% w/w of the composition.

Psoriatic erythroderma involves widespread inflammation and exfoliation of the skin over most of the body surface. It may be accompanied by severe itching, swelling and pain. Therefore, the present invention overcomes these limitations by having very low concentration of alcohol in addition to viscosity modifying agents to provide a skin moistening effect that does not dry up the skin. Further, present invention optionally comprises penetration enhancer in a very low concentration in order to avoid the disruption of the skin lipid structure and hence, prevent significant systemic absorption.

The composition of the present invention further comprises one or more pharmaceutically acceptable excipients selected from, but not limited to lipids, oils, initiators, pH adjusting agents, emollients, humectants, preservatives, antioxidants, penetration enhancer and chelating agents.

Suitable lipids which can be used include one or more of hydrocarbons, fatty alcohols, fatty acids, glycerides or esters of fatty acids with C₁-C₃₆ alkanols. Hydrocarbons may include paraffin or petroleum jelly. Fatty alcohols may include decanol, dodecanol, tetradecanol, hexadecanol or octadecanol. Fatty acids may include C₆-C₂₄ alkanoic acids such as hexanoic acid, octanoic acid, decanoic acid, dodecanoic acid, tetradecanoic acid, hexadecanoic acid, octadecanoic acid, unsaturated fatty acids such as oleic acid and linoleic acid. Glycerides may include olive oil, castor oil, sesame oil, caprylic/capric acid triglyceride or glycerol mono-, di- and tri-esters with palmitic and/or stearic acid. Esters of fatty acids may include C₁-C₃₆ alkanols such as beeswax, carnauba wax, cetyl palmitate, lanolin, isopropyl myristate, isopropyl stearate, oleic acid decyl ester, ethyl oleate and C₆-C₁₂ alkanoic acid esters and the like.

Suitable oils may include one or more of almond oil, apricot seed oil, borage oil, canola oil, coconut oil, corn oil, cotton seed oil, fish oil, jojoba bean oil, lard oil, linseed oil, boiled macadamia nut oil, mineral oil, olive oil, peanut oil, safflower oil, sesame oil, soybean oil, squalane, sunflower seed oil, tricaprylin (1,2,3 trioctanoyl glycerol) and wheat germ oil and the like. The preferred quantity of oil used is in the range of about 5% to about 25% w/w, and more preferably in the range of about 5% to about 20% w/w of the composition.

In a preferred embodiment, the ratio of emulsifying agent to oil in the pharmaceutical composition of the present invention ranges from about 0.1:20 to about 20:0.1, preferably about 0.1:10 to about 10:0.1.

Suitable pH adjusting agents which can be used include one or more of organic or inorganic acids and bases including sodium hydroxide, potassium hydroxide, ammonium hydroxide, phosphate buffers, citric acid, acetic acid, fumaric acid, hydrochloric acid, malic acid, nitric acid, phosphoric acid, propionic acid, sulfuric acid, tartaric acid and the like. In an embodiment, the pH of the composition of the invention may range from about 4.5 to about 7.0, and preferably from 5.0 to about 6.5.

Suitable emollients which can be used include one or more of caprylic/capric triglyerides, castor oil, ceteareth-20, ceteareth-30, cetearyl alcohol, ceteth 20, cetostearyl alcohol, cetyl alcohol, cetyl stearyl alcohol, cocoa butter, diisopropyl adipate, glycerin, glyceryl monooleate, glyceryl monostearate, glyceryl stearate, isopropyl myristate, isopropyl palmitate, lanolin, lanolin alcohol, hydrogenated lanolin, liquid paraffins, linoleic acid, mineral oil, oleic acid, white petrolatum, polyethylene glycol, polyoxyethylene glycol fatty alcohol ethers, polyoxypropylene 15-stearyl ether, propylene glycol stearate, squalane, steareth-2 or -100, stearic acid, stearyl alcohol, urea and the like.

Suitable preservatives which can be used include one or more of phenoxyethanol, parabens (such as methylparaben and propylparaben), propylene glycols, sorbates, urea derivatives (such as diazolindinyl urea), and the like.

Suitable antioxidants which can be used include one or more of ascorbic acid, alpha-tocopherol (vitamin-E), butylated hydroxyanisole (BHA), butylated hydroxytoluene (BHT), glutathione, sodium metabisulphite and the like. The amount of antioxidant may range from about 0.05% to about 1.0% w/w of the total weight of the composition.

Suitable humectants which can be used include one or more of propylene glycol, glycerin, butylene glycol, sorbitol, triacetin and the like.

Suitable chelating agents which can be used include one or more of disodium EDTA, edetate trisodium, edetate tetrasodium, diethyleneamine pentaacetate and the like.

Suitable initiators may include one or more of alcohols like C₁-C₁₂ alcohols, diols and triols, glycerol, methanol, ethanol, propanol, octanol, and the like. The amount of initiator may range from about 3.0% to about 7.0% w/w of the total weight of the composition.

The penetration enhancer is also sometimes called an “absorption enhancer”. A number of penetration enhancers may be utilised including sulfoxides (e.g. DMSO), azones (e.g.laurocapram), pyrrolidones (e.g., 2-pyrrolidone), alcohols and alkanols (e.g., ethanol, decanol, etc.), oleic acid (and derivatives thereof), glycols (e.g., propylene glycol), dimethylformamide (DMF), dimethylacetamide (DMAC), fatty alcohols (e.g., lauryl alcohol), fatty acid esters (e.g. Cetyl myristoleate), fatty acids (e.g. palmitoleic acid), fatty alcohol ethers (e.g., POE(2)-oleyl ether), terpenes, and biologics (e.g., lecithin). Skin penetration enhancers preferably comprise 0.01 to 10.00% w/w of the composition, and more preferably 0.01 to 5.00% w/w.

Suitable dosage form of the invention may include cream, gel, ointment, lotion, lotion, spray and emulsion.

In a preferred embodiment, the composition of the invention is in the form of spray.

In a more preferred embodiment, the present invention relates to a pharmaceutical spray composition comprising low dose of clobetasol and salts or esters thereof in submicron size droplets with oily excipients dispersed in an aqueous medium, said aqueous medium comprising of an emulsifying agent and at least one viscosity modifying agent, agent, wherein the clobetasol and salts or esters thereof is present in concentrations of about 0.005 to 0.025% w/w.

The topical formulations herein described may be applied directly onto affected areas of the skin as a spray. The strength of the formulation and hence the dose of the steroid to be applied will depend on the severity of the disease, the age of the patient, and the extent of the affected area.

The administration could be carried out once or twice daily, preferably twice daily, typically by keeping the spray at a suitable distance from the skin to optimally cover the desired area with one shot.

Further aspects of the present invention also provide dispensing devices for the topical delivery of the formulation onto the skin in the form of sprays. In certain embodiments, the present invention provides devices, into which the formulation is filled, comprising a container, a dispenser and a closure. The closures used for packaging could be made of a polymeric substance such as high-density polyethylene (HDPE), low-density polyethylene (LDPE), or resins. The closures are particularly in the form of caps that are fitted onto the containers to aid in providing support to the dispenser unit and/or to shield the contents of the container from the outside environment. Various container materials include, but are not limited to, tin plated steel, aluminum, stainless steel, plastics, and glass.

An example of a dispenser is a unit containing a pump that can be adapted to fit on any type of container, such as by threads that match threading on the container. The pump is capable of dispensing the sprayable formulations described herein through a dip tube extending into a container from an actuator and attached to a one-way valve, which releases the formulation from an orifice in the actuator in the form of a spray.

The valve may be a metering valve. Various types of valves that can be used include, but are not limited to, continuous spray valves and metering valves. The actuators allow for easy opening and closing of the valve and are an integral part of a package.

Various types of actuators include but are not limited to spray actuators, foam actuators, solid-stream actuators, and special actuators. In another embodiment, a dispensing device may be a device comprising a container, having therein a pouch system or bag containing the product, optionally fitted with a dip tube and an actuator fitted with a valve wherein the container is filled with nitrogen gas or compressed air, surrounding the pouch or bag. Containers can be made of aluminum or tin plate and the pouch system or bag containing the product can be made of layers of polyethylene (PE), polypropylene (PP), polyethylene terephthalate (PET), and aluminum.

The pouch can have a dip tube therein, communicating with the actuator valve, to control the spray rate and reduce droplet size. In other embodiments, a dispensing device useful for dispensing the formulations herein described provides spray rates and spray patterns, in a manner such that substantially uniform dosage is dispensed each time which appreciably covers the desired affected area of the skin onto which the formulation is sprayed.

Devices frequently provide a reproducible distribution of droplets, in distributions where about 90% of the droplets have sizes ranging from about 1 to about 250 microns. The orifice is sized to control the droplet sizes of the dispensed product. The orifice size also affects the provision of a uniform characteristic spray pattern.

The formulations herein described can be applied in an essentially easier and more exact way than creams and ointments can be applied, since for the spray application it is only necessary to spray a given volume, whereas the application of the semi-solid products requires an easily accessible and visual estimation of the cream amount or the ointment amount. Further, smearing and soiling of clothing can more easily be avoided on large surface areas.

For the spray compositions, spreading and rubbing are not necessarily required, contrary to cream and ointment products, since the layer formed on the body surface by evaporation or vaporization of the liquid already has an ideal fine dispersion of active agent.

Administration of the formulation herein described may be indicated for the prophylaxis and/or treatment of a wide range of dermatological diseases or conditions including, but not limited to, contact dermatitis, atopic dermatitis, seborrheic dermatitis, stasis dermatitis, asteatotic eczema, nummular eczema, psoriasis, rosacea, peri-oral dermatitis, scabies, leg ulcers, tuberculous, ringworm or viral skin disease, discoid lupus erythematosus, various forms of lichen such as lichen planus and lichen sclerosus.

Other features of the invention will become apparent in the course of the following descriptions of exemplary embodiments which are given for illustration of the invention and are not intended to be limiting thereof.

### Example-1

| | | Quantity |
|---|---|---|
| Sr. No. | Name of Ingredient | (% w/w) |
| 1 | Clobetasol Propionate | 0.01 |
| 2 | Polyoxyethylenesorbitan monooleate | 3.00 |
| 3 | Dehydrated Alcohol | 5.00 |
| 4 | Glycerin | 5.00 |
| 5 | Soybean Oil | 5.00 |
| 6 | Crosslinked acrylic acid polymers | 1.00 |
| 7 | Sodium Hydroxide | 0.23 |
| 8 | Purified Water | 80.76 |

Process:
1. Clobetasol propionate was dissolved in dehydrated alcohol and polyoxyethylenesorbitan monooleate,
2. Glycerin, soybean oil and purified water was added to the solution of step 1 by stirring,
3. The resulting blend was homogenized to reduce the droplet size to D₉₀ not more than 250 nm with the help of high pressure homogenization to get the nano emulsion,
4. Carbopol was dispersed in purified water under stirring and allowed it to soak for minimum 2 hours,
5. Contents of Step 3 was added to the contents of Step 4 under stirring and homogenized,
6. Sodium hydroxide solution was added to adjust the pH,
7. Bulk of step 6 was filled in proposed packing material

### Example-2

| | | Quantity |
|---|---|---|
| Sr. No. | Name of Ingredient | (% w/w) |
| 1 | Clobetasol Propionate | 0.025 |
| 2 | Polyoxyethylenesorbitan monooleate | 3.00 |
| 3 | Dehydrated Alcohol | 5.00 |
| 4 | Glycerin | 5.00 |
| 5 | Soybean Oil | 5.00 |
| 6 | Crosslinked acrylic acid polymers | 1.00 |
| 7 | Sodium Hydroxide | 0.23 |
| 8 | Purified Water | 80.745 |

Process:
1. Clobetasol propionate was dissolved in dehydrated alcohol and polyoxyethylenesorbitan monooleate,
2. Glycerin, soybean oil and purified water was added to the solution of step 1 by stirring,
3. The resulting blend was homogenized to reduce the droplet size to D₉₀ not more than 250 nm with the help of high pressure homogenization to get the nanoemulsion,
4. Carbopol was dispersed in purified water under stirring and allowed it to soak for minimum 2 hours,
5. Contents of Step 3 was added to the contents of Step 4 under stirring and homogenized,
6. Sodium hydroxide solution was added to adjust the pH,
7. Bulk of step 6 was filled in proposed packing material

### Example-3

| | | Quantity |
|---|---|---|
| Sr. No. | Name of Ingredient | (% w/w) |
| 1 | Clobetasol Propionate | 0.05 |
| 2 | Polyoxyethylenesorbitan monooleate | 3.00 |
| 3 | Dehydrated Alcohol | 5.00 |
| 4 | Glycerin | 5.00 |
| 5 | Soybean Oil | 5.00 |
| 6 | Crosslinked acrylic acid polymers | 1.00 |
| 7 | Sodium Hydroxide | 0.23 |
| 8 | Purified Water | 80.72 |

Process:
1. Clobetasol propionate was dissolved in dehydrated alcohol and polyoxyethylenesorbitan monooleate,
2. Glycerin, soybean oil and purified water was added to the solution of step 1 by stirring,
3. The resulting blend was homogenized to reduce the droplet size to D₉₀ not more than 250 nm with the help of high pressure homogenization to get the nanoemulsion,
4. Carbopol was dispersed in purified water under stirring and allowed it to soak for minimum 2 hours,
5. Contents of Step 3 was added to the contents of Step 4 under stirring and homogenized,
6. Sodium hydroxide solution was added to adjust the pH,
7. Bulk of step 6 was filled in proposed packing material

### Example-4

| | | Quantity |
|---|---|---|
| Sr. No. | Name of Ingredient | (% w/w) |
| 1 | Clobetasol Propionate | 0.001 |
| 2 | Polyoxyethylenesorbitan monooleate | 3.00 |
| 3 | Dehydrated Alcohol | 5.00 |
| 4 | Glycerin | 5.00 |
| 5 | Soybean Oil | 5.00 |
| 6 | Crosslinked acrylic acid polymers | 1.00 |
| 7 | Sodium Hydroxide | 0.23 |
| 8 | Purified Water | 80.769 |

Process:
1. Clobetasol propionate was dissolved in dehydrated alcohol and polyoxyethylenesorbitan monooleate,
2. Glycerin, soybean oil and purified water was added to the solution of step 1 by stirring,
3. The resulting blend was homogenized to reduce the droplet size to D₉₀ not more than 250 nm with the help of high pressure homogenization to get the nanoemulsion,
4. Carbopol was dispersed in purified water under stirring and allowed it to soak for minimum 2 hours,
5. Contents of Step 3 was added to the contents of Step 4 under stirring and homogenized,
6. Sodium hydroxide solution was added to adjust the pH,
7. Bulk of step 6 was filled in proposed packing material

### Example-5

Evaluation of 0.05% and 0.025% Submicron Formulations in IMQ Model

The submicron formulation of 0.05% (Example-3), which is equivalent to the standard marketed clobetasol formulations, and a lower strength of 0.025% (Example-2) of particle size 200 nm each, were prepared and tested in preclinical psoriasis models for their efficacy.

Effect of clobetasol spray was evaluated in an animal model of imiquimod (IMQ)-induced psoriasis in male C57BL6/J mice.

Study:

Animals were divided into six groups, each group comprised of six animals. First three groups received submicron formulation of clobetasol in concentration of
0.025% & 0.05% and the corresponding placebo (100 μl/mouse, topically) respectively. Following two groups received clobex spray 0.05% (Galderma) and the corresponding placebo (100 μl/mouse, topically) respectively.

Formulations were applied topically daily (day1 to 5) to the animals 100 μl/mouse (80 μl on the back & 20 μl on both sides of the ear. Five hour after the treatment, 55 μl (40 μl onto back & 15 μl onto both sides of the ear) of imiquimod (5% IMQ) cream was topically applied on each animal for five days. Basal ear thickness was measured on day 1 and subsequent measurements were on day 4, 5 & 6. Skin erythema and scaling was evaluated daily by visual assessment and grading on a scale of 0-3 based on severity. (0: normal, 1: mild, 2: moderate, 3: severe) On day 6, blood for hematology and serum was collected for analysis of corticosterone. Animals were then sacrificed, and ear punch of 8 mm diameter and spleen was collected and weighed.

Results:

Clobetasol spray sub-micron formulations of 0.05% and 0.025% strengths showed similar inhibition in ear thickness of around 67% and 64% respectively, compared to its placebo. Whereas, Galderma's Clobex spray at 0.05% concentration showed 57% inhibition compared to its placebo (FIG. 1). Inhibition of ear weight was also similar for both the strengths which were comparable for Clobex spray. Clobetasol nano-formulation spray of 0.05% and 0.025% showed around 65% and 51% inhibition in scaling score compared to placebo. While, Clobex spray formulation caused mere 6% inhibition in scaling score. Similarly in erythema score, both the clobetasol nano-formulation treatments resulted in 75-80% inhibition; while that with Clobex spray was only 10% compared to their respective placebos. One observation for the Galderma placebo formulation was that, it was not well tolerated and aggravated the ear scaling associated with thickness. There was inhibition of spleen weight which was similar for all the treatments and varied between 60-68% (FIG. 5).

The formulations were next tested for their HPA axis suppression potential. Both the strengths of clobetasol nano-formulation caused absolute inhibition of circulating corticosterone levels (FIG. 6).

Observations:

Both the clobetasol submicron formulations of 0.05% and 0.025% strengths demonstrated similar efficacy in the IMQ induced psoriasis model. The effects were superior to the marketed Clobex spray from Galderma. It was observed that when Clobex vehicle (without clobetasol) and Clobex itself was applied on the skin of C57BL/6 mice, there were badly discoloured patches on the skin. However, the skin of C57BL/6 mice where the formulation of present invention was applied didn't show any such discoloration.

### Example-6

Evaluation of 0.01% Submicron Formulations in IMQ Model

The submicron formulation of a lower strength 0.01% (Example-1) were prepared and tested in preclinical psoriasis models for their efficacy.

Effect of clobetasol spray was evaluated in an animal model of imiquimod (IMQ)-induced psoriasis in male C57BL6/J mice.

Study:

Animals were divided into three groups, each group comprised of six animals. Formulations were applied topically daily (day1 to 5) to the animals 100 μl/mouse (80 μl onto back & 20 μl onto both sides of the ear. Five hour after the treatment, 55 μl (40 μl onto back & 15 μl onto both sides of the ear) of imiquimod (5% IMQ) cream was topically applied on each animal for five days. Basal ear thickness was measure on day 1 and subsequent measurements were on day 4, 5 & 6. Skin erythema and scaling was evaluated daily by visual assessment and grading on a scale of 0-3 based on severity. (0: normal, 1: mild, 2: moderate, 3: severe) On day 6, blood for hematology and serum was collected for analysis of corticosterone. Animals were then sacrificed, and ear punch of 8 mm diameter and spleen was collected and weighed.

Results:

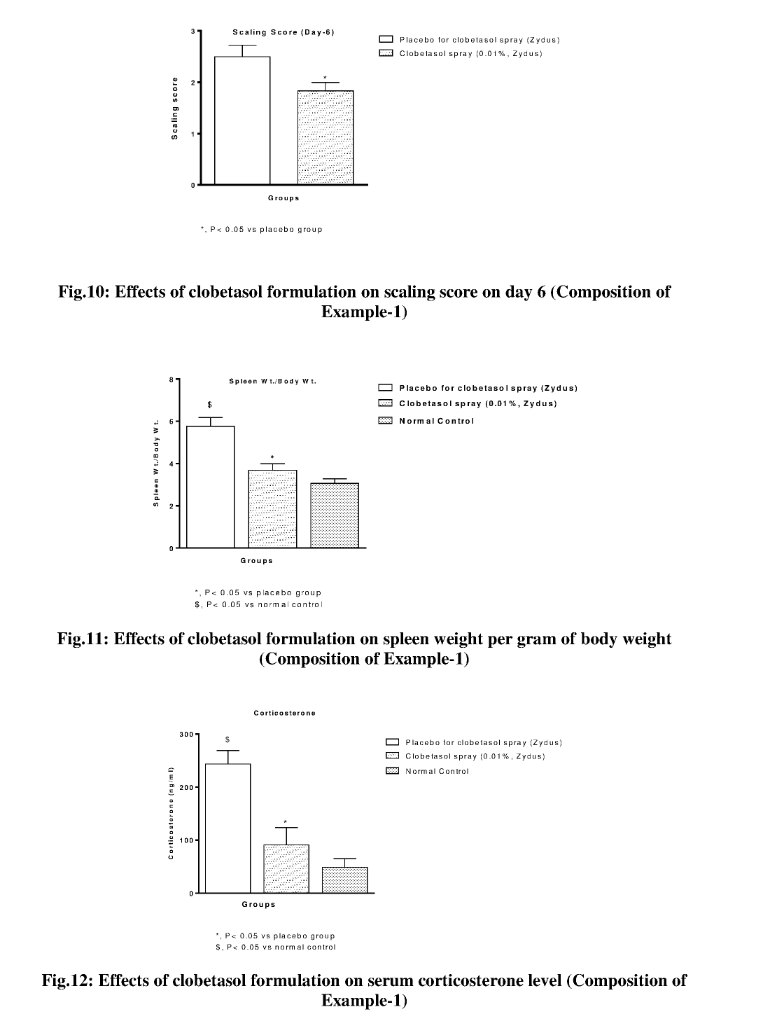

Clobetasol spray submicron formulations of 0.01% (200 nm) strength showed inhibition in ear thickness of around 41%, compared to the placebo (FIG. 7). Clobetasol nano-formulation spray showed around 27% inhibition in scaling score compared to placebo (FIG. 10). In erythema score, the clobetasol nano-formulation treatments resulted in ˜50% inhibition (FIG. 9). There was 36% inhibition of spleen weight compared to the control, as a result of the treatment (FIG. 11). The formulations were next tested for their HPA axis suppression potential. 0.01% formulation brought the IMQ induced elevated cortecosterone levels to normal circulating levels (FIG. 12).

Observations:

The clobetasol submicron formulations of 0.01% strength showed adequate efficacy but did not suppress the corticosterone level below the normal circulating concentrations. This indicated that the 0.01% nano-formulation of clobetasol, which was lower than the marketed 0.05% formulations, was efficacious in preclinical psoriasis model and was devoid of the HPA axis suppression liability.

### Example-7

Evaluation of 0.01% Submicron Formulations in Oxazolone Induced Delayed Type Hypersensitivity Model:

The submicron formulation of 0.01% (Example-1), which demonstrated efficacy in IMQ model was prepared and tested in a second preclinical psoriasis models for its efficacy.

This study was conducted to ensure the efficacy of the 0.01% clobetasol submicron formulation spray.

Effect of clobetasol spray was evaluated in an animal model of oxazolone (OXA)-induced dermatitis in female BALB/c mice.

Study:

Animals were divided into two groups, each group comprised of six animals. On day 1, animals were sensitized through topical application of 100 μl of OXA solution (5 mg/ml in ethanol) on the shaved abdomen. On day 5 & 6, twenty μl of OXA solution (5 mg/ml in acetone) was applied onto the right ear (10 μl onto each side) 30 min prior to the topical administration of placebo or clobetasol spray formulation (10 μl onto each side). Ear thickness was measure on day 5, 6 & 7. On day 7, animals were sacrificed, ear punch of 8 mm diameter collected and weighed.

Results:

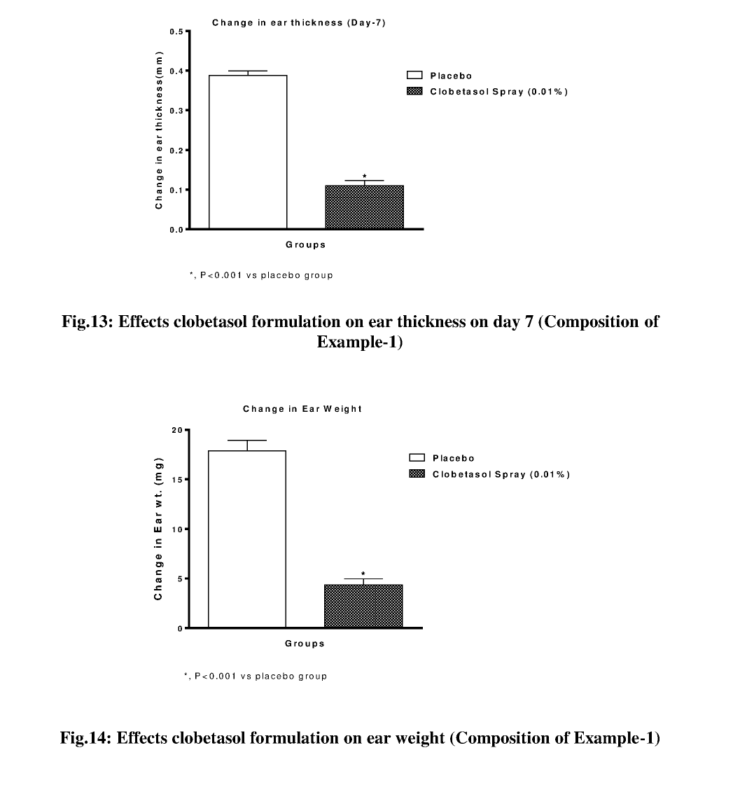

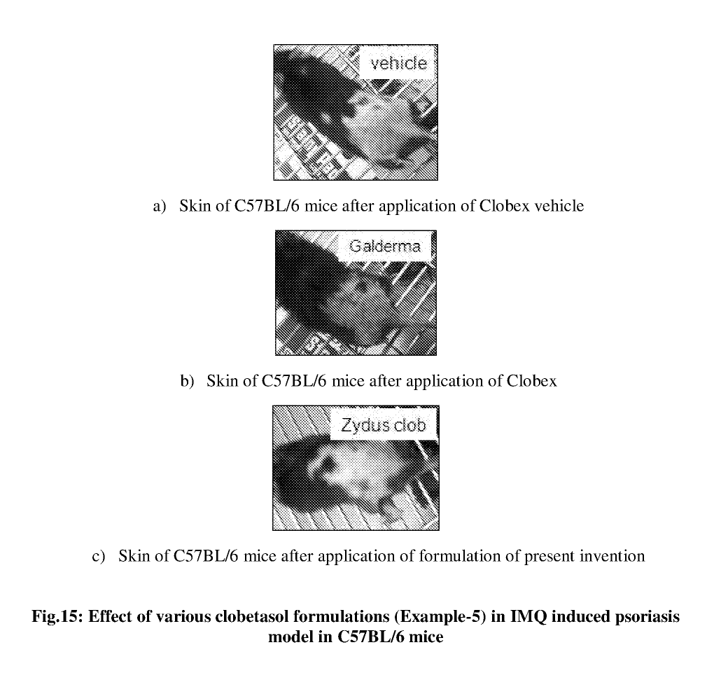

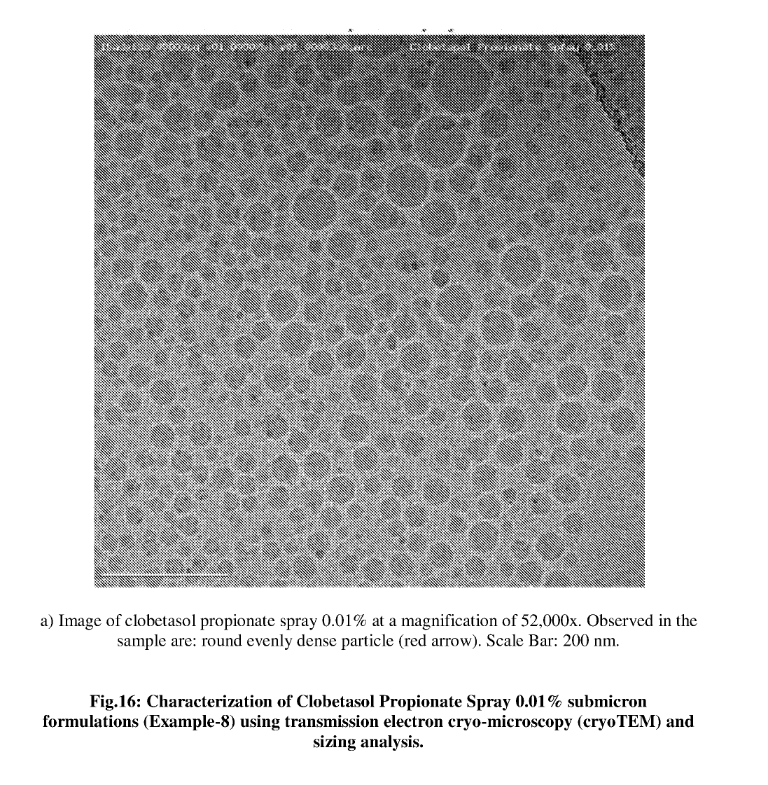

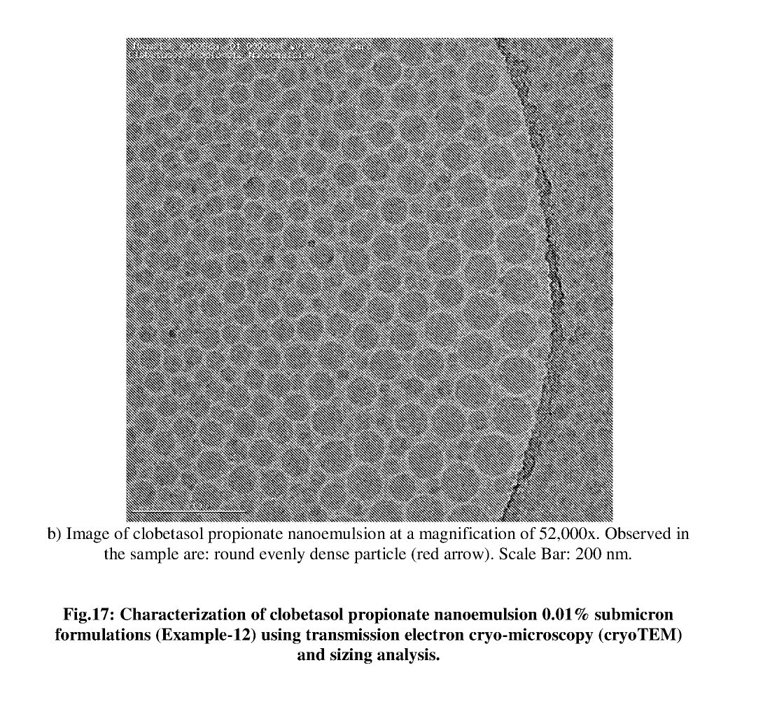

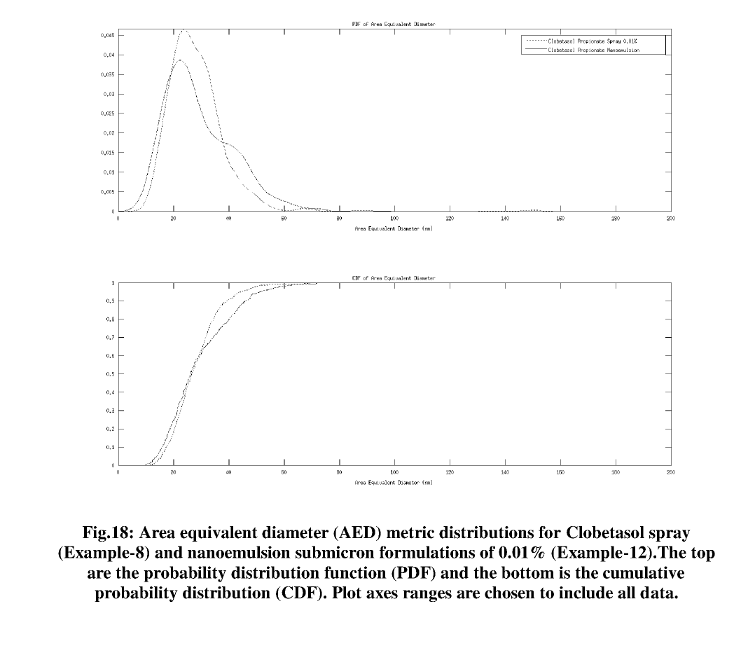

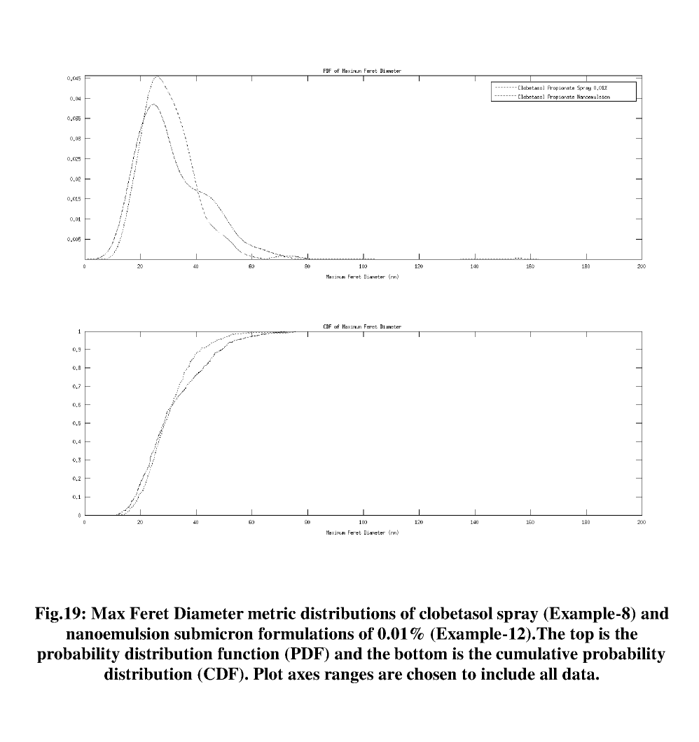

Clobetasol spray submicron formulations of 0.01% showed 71% and 75% inhibition in ear thickness (FIG. 13) and weight (FIG. 14) respectively in comparison to the placebo group.

Observations:

The 0.01% clobetasol submicron formulation of present invention is efficacious and safe as evidenced from the study in two preclinical models of psoriasis.

### Example-8

| | | Quantity |
|---|---|---|
| Sr. No. | Name of Ingredient | (% w/w) |
| 1 | Clobetasol Propionate | 0.01 |
| 2 | Polyoxyethylenesorbitan monooleate | 3.00 |
| 3. | Alcohol | 5.00 |
| 4 | Glycerol | 5.00 |
| 5 | Soybean Oil | 5.00 |
| 6 | Crosslinked acrylic acid polymers | 0.10 |
| 7 | Sodium Hydroxide | 0.013 |
| 8 | Purified Water | 81.877 |

Process:
1. Clobetasol propionate was dissolved in dehydrated alcohol and polyoxyethylenesorbitan monooleate.
2. Glycerol, soybean oil and purified water was added to the solution of step 1 by stirring.
3. The resulting blend was homogenized to reduce the droplet size to D₉₀ not more than 250 nm with the help of high pressure homogenization to get the nanoemulsion.
4. Carbopol was dispersed in purified water under stirring and allowed it to soak for minimum 2 hours.
5. Contents of Step 3 was added to the contents of Step 4 under stirring and homogenized.
6. Sodium hydroxide solution was added to adjust the pH.
7. Bulk of step 6 was filled in proposed packing material

### Example-8

Stability Studies of 0.01% Submicron Formulations of Clobetasol Propionate Spray

| | | | Condition: 40° C./75% RH | | |
|---|---|---|---|---|---|
| Sr. No | Parameters | Initial | 1M | 2M | 3M |
| 1 | Assay of clobetasol | 101.8 | 100.4 | 97.8 | 99.7 |
| | propionate | | | | |
| 2 | Total impurities | 0.45 | 0.43 | 0.28 | 0.5 |
| 3 | pH | 6.25 | 6.09 | 5.82 | 5.85 |
| 4 | Viscosity (cps) | 144.4 | 135.9 | 115.2 | 92.4 |
| 5 | Globule size (in nm) | 150 | 141 | 133 | 138 |

### Example-9

| | | Quantity |
|---|---|---|
| Sr. No. | Name of Ingredient | (% w/w) |
| 1 | Clobetasol Propionate | 0.01 |
| 2 | Polyoxyethylenesorbitan monooleate | 3.00 |
| 3. | Alcohol | 5.00 |
| 4 | Glycerol | 5.00 |
| 5 | Soybean Oil | 5.00 |
| 6 | SEPINEO ™ P 600 | 0.30 |
| 7 | Sodium Hydroxide | 0.013 |
| 8 | Purified Water | 81.677 |

Process:
1. Clobetasol Propionate was dissolved in dehydrated alcohol and polyoxyethylenesorbitan monooleate,
2. Glycerol, soybean oil and purified water was added to the solution of step 1 by stirring,
3. The resulting blend was homogenized to reduce the droplet size to D₉₀ not more than 250 nm with the help of high pressure homogenization to get the nanoemulsion,
4. SEPINEO™ P 600 was dispersed in purified water under stirring and allowed it to soak for minimum 2 hours,
5. Contents of Step 3 was added to the contents of Step 4 under stirring and homogenized,
6. Sodium hydroxide solution was added to adjust the pH,
7. Bulk of step 6 was filled in proposed packing material.

### Example-10

| | | Quantity |
|---|---|---|
| Sr. No. | Name of Ingredient | (% w/w) |
| 1 | Clobetasol Propionate | 0.01 |
| 2 | Polyoxyethylenesorbitan monooleate | 3.00 |
| 3. | Alcohol | 5.00 |
| 4 | Glycerol | 5.00 |
| 5 | Soybean Oil | 5.00 |
| 6 | Hydroxyethyl cellulose | 0.25 |
| 7 | Sodium Hydroxide | 0.013 |
| 8 | Purified Water | 81.727 |

Process:
1. Clobetasol propionate was dissolved in dehydrated alcohol and polyoxyethylenesorbitan monooleate,
2. Glycerol, soybean oil and purified water was added to the solution of step 1 by stirring,
3. The resulting blend was homogenized to reduce the droplet size to D₉₀ not more than 250 nm with the help of high pressure homogenization to get the nanoemulsion,
4. Hydroxyethyl cellulose was dispersed in purified water under stirring and allowed it to soak for minimum 2 hours,
5. Contents of Step 3 was added to the contents of Step 4 under stirring and homogenized,
6. Sodium hydroxide solution was added to adjust the pH,
7. Bulk of step 6 was filled in proposed packing material.

### Example-11

| | | Quantity |
|---|---|---|
| Sr. No. | Name of Ingredient | (% w/w) |
| 1 | Clobetasol Propionate | 0.01 |
| 2 | Polyoxyethylenesorbitan monooleate | 3.00 |
| 3. | Alcohol | 5.00 |
| 4 | Glycerol | 5.00 |
| 5 | Soybean Oil | 5.00 |
| 6 | Hydroxypropylmethyl cellulose | 0.50 |
| 7 | Sodium Hydroxide | 0.013 |
| 8 | Purified Water | 81.477 |

Process:
1. Clobetasol propionate was dissolved in dehydrated alcohol and polyoxyethylenesorbitan monooleate,
2. Glycerol, soybean oil and purified water was added to the solution of step 1 by stirring,
3. The resulting blend was homogenized to reduce the droplet size to D₉₀ not more than 250 nm with the help of high pressure homogenization to get the nanoemulsion,
4. Hydroxypropylmethyl cellulose was dispersed in purified water under stirring and allowed it to soak for minimum 2 hours,
5. Contents of Step 3 was added to the contents of Step 4 under stirring and homogenized,
6. Sodium hydroxide solution was added to adjust the pH,
7. Bulk of step 6 was filled in proposed packing material.

### Example-12

| | | Quantity |
|---|---|---|
| Sr. No. | Name of Ingredient | (% w/w) |
| 1 | Clobetasol Propionate | 0.02 |
| 2 | Polyoxyethylenesorbitan monooleate | 6.00 |
| 3. | Alcohol | 10.00 |
| 4 | Glycerol | 10.00 |
| 5 | Soybean Oil | 10.00 |
| 6 | Purified Water | q.s. |

Process:
1. Clobetasol propionate was dissolved in dehydrated alcohol and polyoxyethylenesorbitan monooleate,
2. Glycerol, soybean oil and purified water was added to the solution of step 1 by stirring,
3. The resulting blend was homogenized to reduce the droplet size to D₉₀ not more than 250 nm with the help of high pressure homogenization to get the nano emulsion,
4. Bulk of step 3 was filled in proposed packing material.

### Example-13

Characterization of 0.01% Submicron Formulations of Clobetasol Propionate Spray (Example-8) and Clobetasol Propionate Nanoemulsion (Example-12) Using Transmission Electron Cryo-Microscopy (cryoTEM) and Sizing Analysis

Study:

Electron microscopy was performed using an FEI Tecnai T12 electron microscope, operating at 120 keV equipped with an FEI Eagle 4K×4K CCD camera. Vitreous ice grids were transferred into the electron microscope using a cryostage that maintains the grids at a temperature below −170° C. Images of each grid were acquired at multiple scales to assess the overall distribution of the specimen. After identifying potentially suitable target areas for imaging at lower magnifications, high magnification images were acquired at nominal magnifications of 110,000× (0.10 nm/pixel), 52,000× (0.21 nm/pixel) and 21,000× (0.50 nm/pixel).

The 52,000× magnification images were further used in the analysis of Particle Size Characterization.

Results:
Clobetasol Propionate Spray 0.01% (Example-8) had a mean area equivalent diameter (AED) of 28.1 nm±10.3 nm and a mean circularity of 0.95.
Particles sized ranged from 9 nm-154 nm in diameter.
Clobetasol propionate spray had an AED D₁₀ of 17.4 nm, D₂₅ of 21.4 nm, Dso₀ of 26.6 nm, D₇₅ of 32.6 nm and D₉₀ of 39.3 nm.
Clobetasol Propionate nanoemulsion (Example-12) had a mean area equivalent diameter (AED) of 29.2 nm±12.2 nm and a mean circularity of 0.92.
Particles sized ranged from 8 nm-93 nm in diameter.
Clobetasol propionate nanoemulsion had an AED D₁₀ of 18.0 nm, D₂₅ of 22.4 nm, D₅₀ of 28.4 nm, D₇₅ of 39.3 nm and D₉₀ of 49.2 nm.

Observations:

Samples of clobetasol spray and 0.01% submicron nanoemulsion showed evenly dense spherical particles. These particles had no visible bilayers and resembled emulsion particles or oil droplets. Particles mostly ranged from 8 nm-120 nm with very few particles >150 nm. Moreover, the particles were well-dispersed and did not tend to clump.

While the invention has been described in terms of its specific embodiments, certain modifications and equivalents will be apparent to those skilled in the art and are intended to be included within the scope of the invention.

We claim:

1. A pharmaceutical composition comprising low dose of corticosteroids and salts or esters thereof in submicron size droplets with oily excipients either alone or dispersed in an aqueous medium, wherein the corticosteroids and salts or esters thereof is present in concentrations of about 0.001% to about 0.05% w/w of the composition.

2. The pharmaceutical composition as claimed in claim 1, wherein the submicron size droplets of corticosteroids and salts or esters thereof have a particle size (D₉₀) of 0.05 to 0.25 microns.

3. The pharmaceutical composition as claimed in claim 1, wherein the corticosteroids and salts or esters thereof comprises one or more of hydrocortisone; hydrocortisone acetate; hydrocortisone butyrate; hydrocortisone valerate; triamcinolone acetonide; fluocinolone acetonide; 16α-hydroxy prednisolone-16α, 17-acetonide; fluohydrocortisone; 1-dehydroxycortisone; 3-methasone; 9α, 11β-dicloro-6α-fluoro-21-hydroxy-16α, 17α-isopropylidenedioxypregna-1, 4-diene-3,20-dione; 9α-fluoro-11β,17α-21-trihydroxy-16β-methylpregna-1, 4-diene-3,20-dione; 9α-fluoro-11β,21-dihydroxy-16β-methyl-17α-valeroxy-pregna-1,4-diene-3,20-dione; 17α,21-dihydroxypregn-4-ene-3,11,20-trione; 17α-hydroxy-21-acetoxypregn-4-ene-3,11, 20-trione; 21-hydroxypregn-4-ene-3, 20-dione; 21-acetoxypregn-4-ene-3, 20-dione; 21-divaloxypregn-4-ene-3, 2-dione; 9α-fluoro-11β, 17α,21-trihydroxy-16α-methylpregna-1,4-diene-3, 20-dione; 6α,90-difluoro-11β, 17α-dihydroxypregna-1,4-diene-3, 20-dione; 6α-fluoro-11β,21-dihydroxy-16-17α-isopropylidenedioxypregn-4-ene-3, 20-dione; 11β,17α-dihydroxy-21-acetoxypregna-4-ene-3, 20-dione; 6α-methyl-11β,17α,21-trihydroxypregna-1,4-diene-3, 20-dione; 6α-methyl-11β,17α-dihydroxy-21-acetoxy-pregna-1,4-diene-3, 20-dione; 6α-fluoro-11β,17α,21-trihydroxy-16α-methylpregna-1, 4-diene-3, 20-dione; 6α-fluoro-11β,17α-dihydroxy-16α-methyl-21-acetoxy-pregna-1,4-diene-3, 20-dione; 6α-fluoro-11β,hydroxy-16α, 17α-isopropylidenedioxy-21-acetoxypregna-1,4-diene-3,20-dione; 11β,17α,21-trihydroxypregna-1,4-diene-3,20-dione; 11β,17α-dihydroxy-21-acetoxypregna-1,4-diene-3, 20-dione; 17α,21-dihydroxypregna-1,4-diene-3,11,20-trione; 17α,hydroxy-21-acetoxypregna-1,4-diene-3-11,20-trione; 9α-fluoro-11β,16β,17α,21-tetrahydroxypregna-1,4-diene-3, 20-dione; 9α-fluoro-11β,16α, 17-trihydroxy-21-acetoxypregna-1,4-diene-3, 20-dione; 6α,9α-difluoro-11β,21-dihydroxy-16α-methyl-17α-valeroxypregna-1,4-diene-3, 20-dione; 6α,9α-difluoro-11β,17α,21-trihydroxy-16α-methyl pregna-1,4-diene-3, 20-dione; 6α,7α-difluoromethylene-11β,17α,21-trihydroxypregna-4-ene-3, 20-dione; 6α,9α-difluoro-11β-hydroxy-16α, 17α-isopropylidenedioxy-21-chloropregna-1,4-diene-3, 20-dione; 9α, 11β-dichloro-6α,21-difluoro-16α, 17α-isopropylidenedioxypregna-1,4-diene-3,20-dione, and 9α, 11β,21-trichloro-6α-fluoro-16α,17α-isopropylidenedioxypregna-1,4-diene-3, 20-dione; clobetasol and salts or esters thereof.

4. The pharmaceutical composition as claimed in claim 1, wherein the composition comprises submicron size droplets of clobetasol or salts, or esters thereof.

5. The pharmaceutical composition as claimed in claim 1, wherein the composition comprises another active agent in combination with clobetasol and salts or esters thereof.

6. The pharmaceutical composition as claimed in claim 5, wherein the another active agent comprises one or more of a steroid, non-steroidal anti-inflammatory drug, antibiotic, tranquilizer, sedative, anti-histaminic, antifungal, antibacterial, antiviral, disinfectant, antipsoriatic agent or a local anesthetic.

7. The pharmaceutical composition as claimed in claim 1, wherein the composition comprises at least one viscosity modifying agent.

8. The pharmaceutical composition as claimed in claim 7, wherein the at least one viscosity modifying agent comprises one or more of cellulose polymer, a cellulose derivative, crosslinked acrylic acid polymers (acrylamide/sodium acryloyldimethyltaurate copolymer-isohexadecane-polysorbate 80 or “carbomer”), polyvinyl alcohol, poloxamers, polysaccharides, polyvinyl pyrrolidone, and xanthan gum.

9. The pharmaceutical composition as claimed in claim 7, wherein the at least one viscosity modifying agent is present in an amount of from about 0.01 to 5% w/w of the composition.

10. The pharmaceutical composition as claimed in claim 1, wherein the composition comprises at least one emulsifying agent.

11. The pharmaceutical composition as claimed in claim 10, wherein the at least one emulsifying agent comprises one or more of lecithin; mixture of about 70% of phosphatidylcholine, 12% phosphatidylethanolamine and about 15% other phospholipids; mixture comprising about 60% phosphatidylcholine, 18% phosphatidylethanolamine and 12% other phospholipids; a purified phospholipid mixture; phospholipid mixture comprising about 80% phosphatidylcholine, 8% phosphatidylethanolamine, 3.6% non-polar lipids and about 2% sphingomyelin, polyoxyethylene sorbitan ester, copolymer of polyoxyethylene and polyoxypropylene.

12. The pharmaceutical composition as claimed in claim 11, wherein the at least one emulsifying agent s present in an amount of from about 0.1% to 10% w/w of the composition.

13. A pharmaceutical composition comprising low dose of clobetasol and salts or esters thereof in submicron size droplets with oily excipients dispersed in an aqueous medium, the aqueous medium comprising an emulsifying agent and at least one viscosity modifying agent, wherein the clobetasol and salts or esters thereof is present in concentrations of about 0.005 to 0.025% w/w.

14. The pharmaceutical composition as claimed in claim 1, wherein the composition further comprises one or more pharmaceutically acceptable excipients selected from lipids, oils, initiators, pH adjusting agents, emollients, humectants, preservatives, antioxidants, penetration enhancer and chelating agents.

15. The pharmaceutical composition as claimed in claim 1, wherein the composition is in the form of a cream, a gel, an ointment, a lotion, a spray, or an emulsion.

16. A pharmaceutical compositions comprising low dose of corticosteroids and salts or esters in concentrations of about 0.005 to about 0.025% w/w, wherein the process of preparation comprising following steps:
a) combining an oily phase comprising corticosteroids and salts or esters thereof with other pharmaceutically acceptable excipients with an aqueous phase to form an emulsion;
b) reducing the droplet size of emulsion of step a) to a droplet size having D₉₀ particle size of less than 250 nm; and
c) mixing other pharmaceutically acceptable excipients to the emulsion obtained in step b) and converting it into a suitable finished dosage form.
